(19) United States
(12) Patent Application Publication
(10) Pub. No.: US 2015/0292032 A1
(43) Pub. Date: Oct. 15, 2015

(54) TREATMENT OF CANCER BY TARGETING QUIESCENT CANCER CELLS

(71) Applicant: FELICITEX THERAPEUTICS, INC., Newton, MA (US)

(72) Inventors: Maria Vilenchik, Newton, MA (US); Michael Frid, Medford, MA (US); Yuri Gankin, Newtown, MA (US)

(73) Assignee: FELICITEX THERAPEUTICS, INC., Newton, MA (US)

(21) Appl. No.: 14/679,048

(22) PCT Filed: Oct. 10, 2013
(86) PCT No.: PCT/US13/64345

§ 371 Date: Apr. 6, 2015

Related U.S. Application Data
(60) Provisional application No. 61/712,079, filed on Oct. 10, 2012.
(60) Provisional application No. 61/843,565, filed on Jul. 8, 2013.

(51) Int. Cl. C12Q 1/68; G01N 33/574; A61K 31/519; A61K 31/4192; C07D 249/18; A61K 38/08; C07K 7/06; C07D 471/04; C07D 473/16; A61N 5/10; A61K 31/52

(52) U.S. Cl. CPC C12Q 1/6886; A61N 5/10; G01N 33/57496; A61K 31/519; A61K 31/4192; A61K 31/52; A61K 38/08; C07K 7/06; C07D 471/04; C07D 473/16; C07D 249/18; G01N 2333/91205; C12Q 2600/158

(57) ABSTRACT

A method for treating a subject suffering from a neoplasm, comprising determining the expression level of a DYRK1 selected from DYRK1A or DYRK1B within a cell population in a sample of neoplastic cells and determining the quiescent state of these cells, and if the DYRK1 is expressed in the sample then administering an effective amount of a modulator of DYRK1 activity, either alone or as a part of a combination therapy.

### RELATED APPLICATIONS

This application is the U.S. national phase application of International Application Number PCT/US2013/064345 filed in the U.S. on Oct. 10, 2013 and which claims the benefit of U.S. Provisional Application No. 61/712,079, filed on Oct. 10, 2012 and U.S. Provisional Application No. 61/843,565, filed on Jul. 8, 2013. The entire teachings of the above applications are incorporated herein by reference.

### FIELD OF THE INVENTION

This invention relates to the field of diagnostic testing for and treatment of cancer and other conditions, and more particularly to diagnostics related to DYRK1 kinase expression and quiescent cancer cell monitoring, and to treatments related to DYRK1 modulation.

### BACKGROUND OF THE INVENTION

Cancer cell quiescence, effectively cancer cell sleep state, has been recognized recently as a major mechanism of cancer resistance to treatments and a pathway for cancer recurrence. This quiescence, alternatively called cancer cellular dormancy, is due to arrest at G₀. Typically, a cell enters a mitotic cycle from gap phase 1 (G₁), as shown in FIG. 1. After a synthesis phase (S) and a short pre-mitotic interval (G₂), the cell divides by mitosis (M) followed by a return to G₁. Instead of G₁, however, a cell can enter dormancy (or quiescence) phase G₀.

A quiescent state is reversible and can be inherent to a cell type or brought about by environmental factors such as lack of growth factors or nutrients, or induced as in pharmacological quiescence. Cells can remain in the quiescent state for an indeterminate period of time, until receipt of a signal to enter the cell division cycle. A quiescent cancer cell in a G₀ state is not susceptible to chemotherapy because conventional chemotherapy or radiation treatments target dividing cells. Typically, dividing cells are targeted by damaging exposed DNA, interfering with mitosis, or other mechanisms. The energy and nutrient requirements of a quiescent cancer cell are reduced relative to a dividing cancer cell. Therefore, quiescent cancer cells are insensitive to antiangiogenic therapies.

DYRK1B, dual specificity tyrosine-phosphorylation-regulated kinase 1B, also called Mirk or minibrain-related kinase, is expressed in cells of multiple cancers including but not limited to pancreatic, colon, ovarian, breast, lung cancers, osteosarcomas, and hematopoietic cancers. DYRK1B is not expressed in the corresponding healthy tissues at detectable levels. Recent studies have suggested that DYRK1B is essential for allowing quiescent cancer cells to stay in the quiescent state, thus promoting cancer cell survival. The expression or overexpression of DYRK1B in cancer cells indicates that cancer is dependent on cancer cell quiescence as a survival mechanism.

### SUMMARY OF THE INVENTION

In one example embodiment, the present invention is based on the discovery that DYRK1 kinases are important for the maintenance of cancer cells in a quiescent state. More specifically, Applicant has discovered that inhibition of a DYRK1 kinase blocks the ability of cancer cells to enter into or accumulate in the quiescent state of the cell cycle (FIGS. 5 and 6). Based on this discovery, methods of treatment and modulators of DYRK1 activity are disclosed.

Accordingly, in one example embodiment, the present invention is a method for treating a patient suffering from a neoplasm. The method comprises (a) determining whether a DYRK1 selected from DYRK1A or DYRK1B is expressed in a patient sample; (b) determining whether quiescent neoplastic cells are present in the patient sample; and (c) if the DYRK1 is expressed in the patient sample and if quiescent neoplastic cells are present in the patient sample, then administering to the patient an effective amount of a modulator of DYRK1 activity.

In another example embodiment, the present invention is a method for treating a patient suffering from a neoplasm. The method comprises (a) determining whether a DYRK1 selected from DYRK1A or DYRK1B is expressed in an initial patient sample, the initial patient sample collected from a patient; (b) determining a fraction of quiescent neoplastic cells present in the initial patient sample; (c) if the DYRK1 is expressed in the initial patient sample and if the fraction of quiescent neoplastic cells in the initial patient sample is greater than zero, then administering to the patient a first effective amount of a modulator of DYRK1 activity; (d) determining whether the DYRK1 selected from DYRK1A or DYRK1B is expressed in a subsequent patient sample, the subsequent patient sample collected from the patient after administering to the patient the first effective amount of the modulator of DYRK1 activity; (e) determining a fraction of quiescent neoplastic cells in the subsequent patient sample; and (f) if DYRK1 is expressed in the subsequent patient sample and if the fraction of quiescent neoplastic cells in the initial patient sample is greater then the fraction of quiescent neoplastic cells in the subsequent patient sample, then administering to the patient a second effective amount of a modulator of the DYRK1 activity.

The present invention relates to methods of treatment for subjects suffering from cancer, especially solid and hematopoietic cancers, Down syndrome, early onset Alzheimer's disease and replication of HIV-1 (which can be useful in treatment of AIDS).

The invention is directed to methods of reducing the fraction of quiescent cells in a neoplastic cell population, comprising incubating the neoplastic cell population comprising quiescent cells with a modulator of DYRK1 activity for a period of time sufficient to reduce the fraction of quiescent cancer cells in the cell population. The present invention further relates to methods for determining the presence of quiescent or dormant cancer cells by defining the distribution of the cellular population within a cell cycle (based on their RNA/DNA content) of the particular cancer cell and measuring the expression level of DYRK1B and/or DYRK1A proteins within that particular cell.

In other example embodiments, the present invention is a modulator of DYRK1 activity for use in the treatment of a neoplasm in a patient, wherein the treatment includes determining whether a DYRK1 selected from DYRK1A or DYRK1B is expressed in a patient sample and determining whether quiescent neoplastic cells are present in the patient sample, and if the patient expresses a DYRK1 selected from DYRK1A or DYRK1B and if quiescent neoplastic cells are present in the patient sample, administering an effective amount of the modulator.

In further embodiments of this invention, a subsequent patient sample is collected after the modulator is administered. The subsequent patient sample is analyzed to determine whether a DYRK1 selected from DYRK1A or DYRK1B is expressed in the subsequent patient sample, as well as to determine a fraction of quiescent neoplastic cells in the subsequent patient sample. If the fraction of quiescent neoplastic cells present in the patient sample is greater than the fraction of quiescent neoplastic cells in the subsequent patient sample, and if the patient expresses a DYRK1 selected from DYRK1A or DYRK1B in the subsequent patient sample, then a second effective amount of the modulator is administered.

In another example embodiment, the invention relates to modulators of DYRK1 activity for use in the treatment of a neoplasm in a patient, wherein the patient has been determined to express a DYRK1 selected from DYRK1A or DYRK1B in a patient sample and wherein quiescent neoplastic cells have been determined to be present in the patient sample.

In another example embodiment, the invention relates to modulators of DYRK1 activity for use in the treatment of a neoplasm in a patient, wherein the patient has been determined to express a DYRK1 selected from DYRK1A or DYRK1B in an initial patient sample and wherein a fraction of quiescent neoplastic cells has been determined in the initial patient sample, the treatment including administering to the patient a first effective amount of the modulator, and further wherein the patient has been determined to express a DYRK1 selected from DYRK1A or DYRK1B in a subsequent patient sample and wherein a fraction of quiescent neoplastic cells has been determined to be present in the subsequent patient sample, the subsequent patient sample having been collected after administering a first effective amount of the modulator, and wherein the fraction of quiescent neoplastic cells in the initial patient sample is greater than the fraction of quiescent neoplastic cells in the subsequent patient sample.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram of a mitotic cycle of a eukaryotic cell.

FIG. 2 is a schematic diagram of a mitotic cycle of a eukaryotic cancer cell annotated to indicate the stages of the cell cycle upon which the available chemotherapeutic agents are believed act.

FIG. 3A and FIG. 3B collectively present schematic illustrations of the role DYRK1B is believed to play in the eukaryotic cancer cell progression through G₀/G₁ checkpoint. FIG. 3A is a schematic diagram showing the regulation of p27 and cyclin D1 by the activity of DYRK1B kinase. FIG. 3B is a schematic diagram of a cell exiting the quiescent state and re-entering the cell cycle upon inhibition of DYRK1B with inhibitor.

FIG. 4 is a schematic diagram representing an embodiment of a method of the present invention.

FIG. 5 presents two histograms, each showing the distribution of cell populations within the cell cycle according to their DNA and/or RNA content distribution, as measured by flow cytometry. Panel A shows a histogram of DNA and RNA content distribution in a population of untreated human pancreatic carcinoma (PANC1) cells that express DYRK1B, after 2 days of serum starvation. Panel B shows a histogram of DNA and RNA content distribution in a population of human pancreatic carcinoma (PANC1) cells that express DYRK1B, after 2 days of serum starvation and treatment with an inhibitor of DYRK1B.

FIG. 6 presents two histograms, each demonstrating DNA content distribution in a population of cycling, log-phase human colon cancer (SW620) cells that express DYRK1B, determined by flow cytometry. Panel A shows a histogram of DNA content distribution in a population of untreated cycling, log-phase human colon cancer (SW620) cells. Panel B shows a histogram of DNA content distribution in a population of cycling, log-phase human colon cancer (SW620) cells that express DYRK1B, treated with an inhibitor of DYRK1B.

FIG. 7 shows the structure of the DYRK1B small molecule inhibitor R.

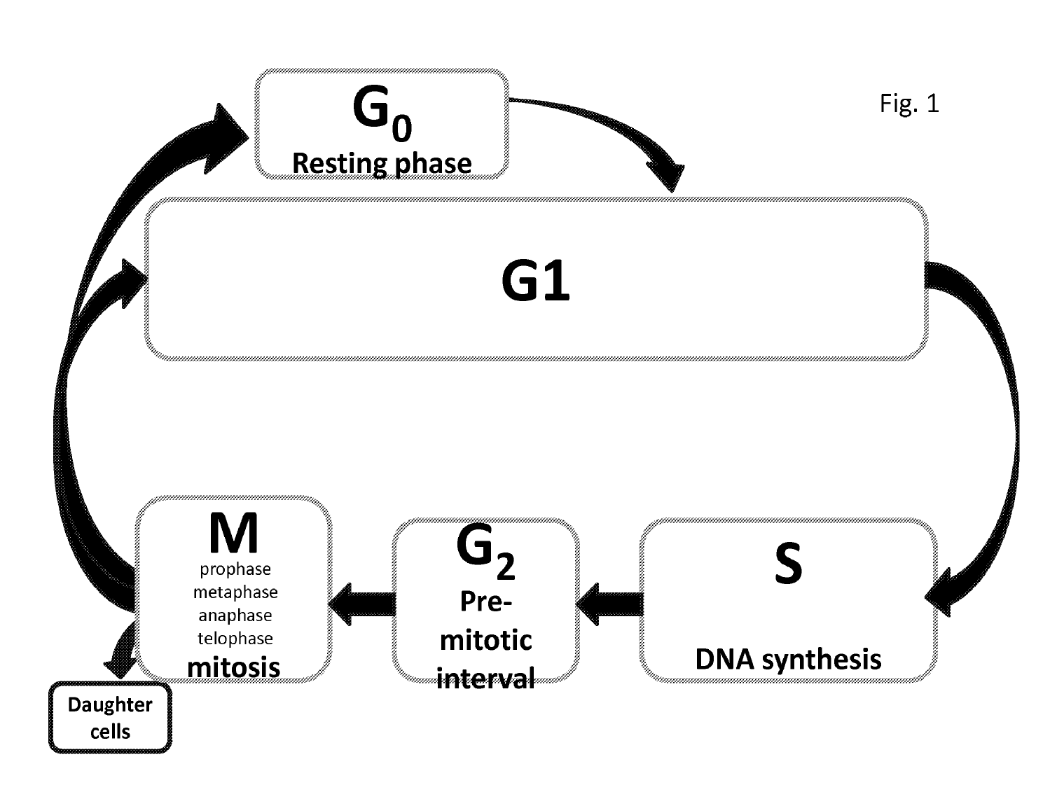

### DETAILED DESCRIPTION OF THE INVENTION

A description of example embodiments of the invention follows.

### Glossary

As used herein, the terms “treat,” “treating,” or “treatment,” mean to counteract a medical condition (e.g., cancer) to the extent that the medical condition is improved according to a clinically-acceptable standard. Improvement of cancer can include: 1) reduced or arrested tumor growth, 2) tumor shrinkage, 3) remission, 4) reduction in metastases, and 5) prevention of recurrence or delay in recurrence. In certain embodiments of the invention, treating includes achieving, partially or substantially, one or more of the following results: partially or totally reducing the cancer mass or the malignant cell count; ameliorating or improving a clinical symptom or indicator associated with solid cancers, hematopoietic cancers, early onset Alzheimer, or Down Syndrome; delaying, inhibiting or preventing the progression of solid cancers, hematopoietic cancers, early onset Alzheimer, or Down Syndrome; or partially or totally delaying, inhibiting or preventing the onset or development of solid cancers, hematopoietic cancers, early onset Alzheimer, or Down Syndrome.

Treating includes prophylactic treatment. “Prophylactic treatment” refers to treatment before clinical symptoms of a target disorder to prevent, inhibit or reduce its occurrence.

As used herein, an “effective amount” refers to an amount of a therapeutic agent or a combination of therapeutic agents that is therapeutically or prophylactically sufficient to treat the target disorder. Examples of effective amounts typically range from about 0.0001 mg/kg of body weight to about 500 mg/kg of body weight. An example range is from about 0.0001 mg/kg of body weight to about 5 mg/kg. For example, the effective amount can range from about 0.005 mg/kg to about 500 mg/kg. In other examples, the range can be from about 0.0001 mg/kg to about 5 mg/kg. In still other examples, effective amounts range from about 0.01 mg/kg of body weight to 50 mg/kg of body weight, or from 0.01 mg/kg of body weight to 20 mg/kg of body weight.

As used herein, the term “subject” refers to a mammal, for example a human, but can also mean an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein a “neoplasm” means an abnormal mass of tissue that results from neoplasia. “Neoplasia” means a process of an abnormal proliferation of cells. In some embodiments of the invention, a neoplasm is a solid cancer, or alternately a hematopoietic cancer. The neoplasia may be benign, pre-malignant, or malignant. The term neoplasm encompasses mammalian cancers, in some embodiments, human cancers, and carcinomas, sarcomas, adenocarcinomas, osteosarcomas, lymphomas, leukemias, including solid and lymphoid cancers, kidney, breast, lung, kidney, bladder, colon, ovarian, prostate, rectal, pancreatic, stomach, brain, head and neck, skin, uterine, testicular, esophagus, and liver cancers, including hepatocarcinoma, lymphoma, including non-Hodgkin's lymphomas (e.g., Burkitt's, Small Cell, and Large Cell lymphomas) and Hodgkin's lymphoma, leukemia, and multiple myeloma.

As used herein, the phrase “patient sample” refers to a sample that includes a cell population taken from the patient, for example during a biopsy or collection of a blood sample.

As used herein, the phrase “determining the expression level of DYRK1” refers to either one or all appropriate: determination of the protein expression or determination of the number of transcripts (gene copy, cDNA, mRNA, or otherwise suitable) using any standard applicable techniques.

As used herein, the term “modulator” means a composition that affects the activity of an enzyme. An example of a modulator is an inhibitor. The term “inhibitor” means any composition that reduces the activity of an enzyme. An example of an inhibitor is a chemical molecule. A measure of the potency of an inhibitor is its “inhibitory concentration 50%” (IC50). IC50 is the concentration of an inhibitor at which 50% of the enzymatic activity is inhibited by the inhibitor.

The term “specific inhibitor” refers to a compound with high specificity for its target (e.g. for DYRK1B or DYRK1A). Specificity of a particular inhibitor is defined as a ratio of the IC50 values of the particular inhibitor for the target of interest versus another target. For example, a kinase inhibitor that is specific for DYRK1A will have an IC50 value for target A (e.g, DYRK1A) lower than that for target B (e.g, DYRK1B). For example, the IC50 value for target A is at least 10 times lower than the IC50 value of the same inhibitor for target B. In other examples, the IC50 value for target A is 100 times lower, or in other example is 1000 times lower. In still other example, the IC50 value for target A is 10,000 times lower than the IC50 value of the same inhibitor for target B.

A “quiescent neoplastic cell”, alternately referred to as a “quiescent cancer cell” means a cancer cell which exists in the quiescent, or G₀, state of the cell cycle. In the G₀ state, cells do not undergo cell division. A “fraction of quiescent neoplastic cells”, as used herein, means the portion of a cell population that exists in the G₀ state of the cell cycle. Determining the fraction of quiescent neoplastic cells includes characterizing a cell population by distribution of its constituent cells within the stages of the cell cycle. The fraction of cells in the G₀ state (i.e., quiescent neoplastic cells) is quantified relative to the total cell population in a sample. The fraction may be expressed as a percentage of the total cell population (i.e. (number of quiescent cells divided by total cells in cell population) multiplied by 100). Characterization of the cell population by distribution of its constituent cells within the stages of the cell cycle may be achieved by techniques known to persons of ordinary skill in the art, and may include analysis by DNA and/or RNA content using flow cytometry methods, for example fluorescence-activated cell sorting (FACS).

In certain embodiments of the invention, a therapeutic agent is a cytotoxic or chemotherapeutic agent or personalized medicine anti-cancer agents. Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, toposimerase inhibitors and others). Further examples include anthracyclines, plant alkaloids, cytostatic agents, cytotoxic agents, immunological modifiers such as interferons, interleukins, growth hormones or other cytokines. Exemplary agents include Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptothecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, Idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixantrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfin, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents.

As used herein, “selecting a therapy” can include detecting quiescent neoplastic cells in a patient sample, and/or confirming the expression of a DYRK1 selected from DYRK1A or DYRK1B in the patient sample. If the cell population from the sample collected from the subject contains quiescent cells that express or overexpress a DYRK1 selected from DYRK1A or DYRK1B, a therapy may be selected that includes administering a modulator of DYRK1 activity.

In further embodiments, “selecting a therapy” also refers to comparing the fraction of quiescent cells in the cell population from a sample collected from a subject to a fraction of quiescent cells observed in a reference cell population, and/or comparing the expression level of a kinase selected from DYRK1B and DYRK1A expressed in the cell population from a sample collected from a subject to the expression level of DYRK1B and/or DYRK1A expressed in a reference cell population. If the cell population from the sample collected from the subject contains quiescent cells and is expressing or overexpressing DYRK1A or DYRK1B, a therapy may be selected that includes administering a DYRK1A or DYRK1B modulator.

### Methods of the Invention

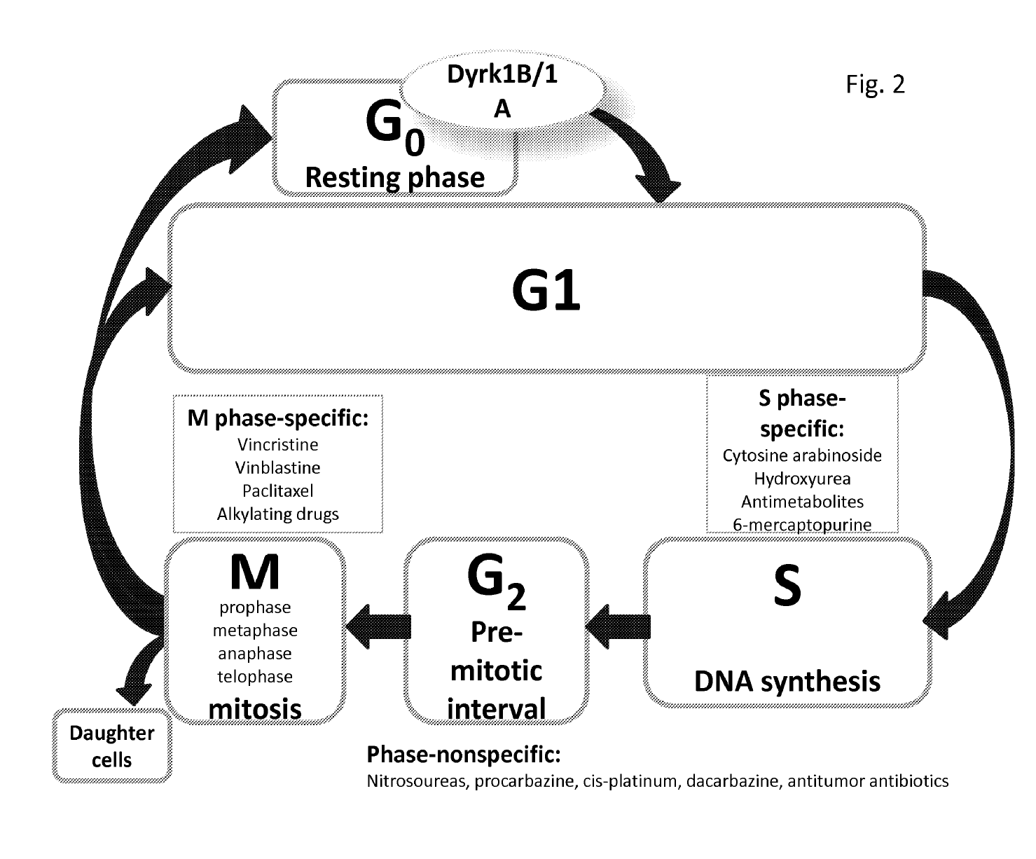

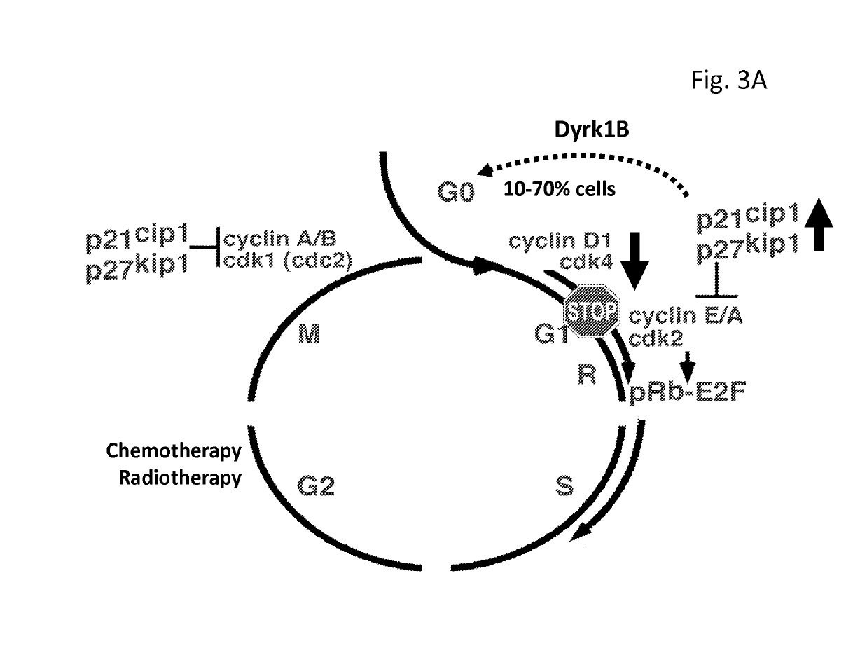

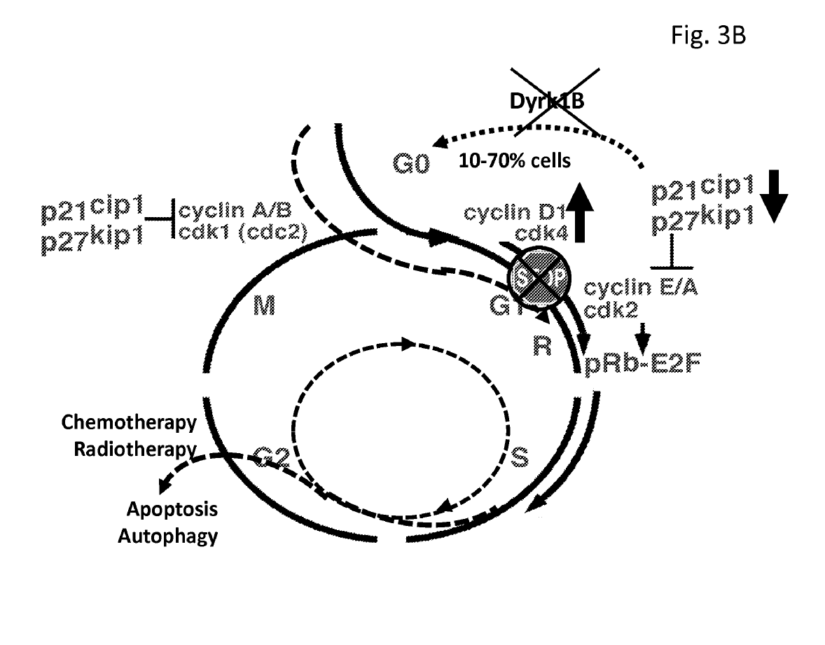

The present invention is based on the discovery illustrated in FIG. 2, FIG. 3A, and FIG. 3B. FIG. 2 is a schematic diagram of a mitotic cycle of a eukaryotic cancer cell annotated to indicate the stages of the cell cycle upon which the available chemotherapeutic agents act. It is illustrated that DYRK1 kinases such as DYRK1A and DYRK1B are important for the maintenance of cancer cells in G₀ state. As shown on FIG. 3A, an increase in DYRK1B expression results in phosphorylation of the specific residues on cyclin D1 (Thr286) and p27 (Ser10) leading to their degradation and increased stability, respectively. Such changes of the total levels of cyclin D1 and p27 proteins result in G₀ arrest. Recent studies have demonstrated that, in certain cancers, a significant percentage of cancer cells exist outside the cell cycle in a reversible G₀ or G₀-like state (Stanton, K. J., Sidner, R. A., Miller, G. A., Cummings, O. W., Schmidt, C. M., Howard, T. J., and Wiebke, E. A. (2003) Analysis of Ki-67 antigen expression, DNA proliferative fraction, and survival in resected cancer of the pancreas, American Journal of Surgery 186, 486-492). Quiescent cancer cells are resistant to cytotoxic drugs that target dividing cells, which allows them to repopulate a cancer. A G₀ state is distinct from a G₁ state, and is maintained by a specific program of gene expression. As shown in FIG. 2, one factor which allows the prolonged stay of cancer cells in quiescent state in vivo is DYRK1B. It is illustrated on FIG. 3B that inhibition of the activity of the kinase DYRK1B results in decreased phosphorylation of the specific corresponding residues on cyclin D1 (Thr286) and p27 (Ser10) resulting in differential changes in the expression of cyclin D1 and p27, stabilization and degradation, respectively. These changes of cyclin D1 and p27 expression levels facilitate cells' progression from G₀ into the active cell cycle.

DYRK1B/Mirk is a member of the Minibrain/DYRK family of kinases which mediates survival and differentiation in certain normal tissues. (Kentrup, H., Becker, W., Heukelbach, J., Wilmes, A., Schurmann, A., Huppertz, C., Kainulainen, H., and Joost, H. G. (1996) Dyrk, a dual specificity protein kinase with unique structural features whose activity is dependent on tyrosine residues between subdomains VII and VIII, J Biol Chem 271, 3488-3495; Becker, W., Weber, Y., Wetzel, K., Eirmbter, K., Tejedor, F. J., and Joost, H. G. (1998) Sequence characteristics, subcellular localization, and substrate specificity of DYRK-related kinases, a novel family of dual specificity protein kinases, Journal of Biological Chemistry 273, 25893-25902). DYRK1B is expressed at detectable levels in skeletal muscle cells and testes. Knockout of DYRK1B caused no evident phenotype in mice even in developing muscle, suggesting that DYRK1B is not an essential gene for normal development. Supporting this interpretation, normal fibroblasts exhibited no alteration in survival after 20-fold depletion of DYRK1B kinase levels.

Thus, DYRK1B does not appear to be an essential gene for survival of normal cells but is essential for the survival of certain cancer cells in patients undergoing treatment with chemotherapeutic or cytotoxic therapies. DYRK1B is expressed or upregulated in certain malignant cancer cells in which DYRK1B mediates survival by retaining cancer cells in quiescent state. These unusual characteristics suggest that DYRK1B may be a selective target for therapeutic intervention. In addition, elevated expression levels of DYRK1B in cells of tissues that, when healthy, do not express DYRK1B at detectable levels will be a diagnostic marker for identifying resting cancers that would lead to cancer resistance and insensitivity to chemotherapy and later result in cancer growth, recurrence, and metastasis.

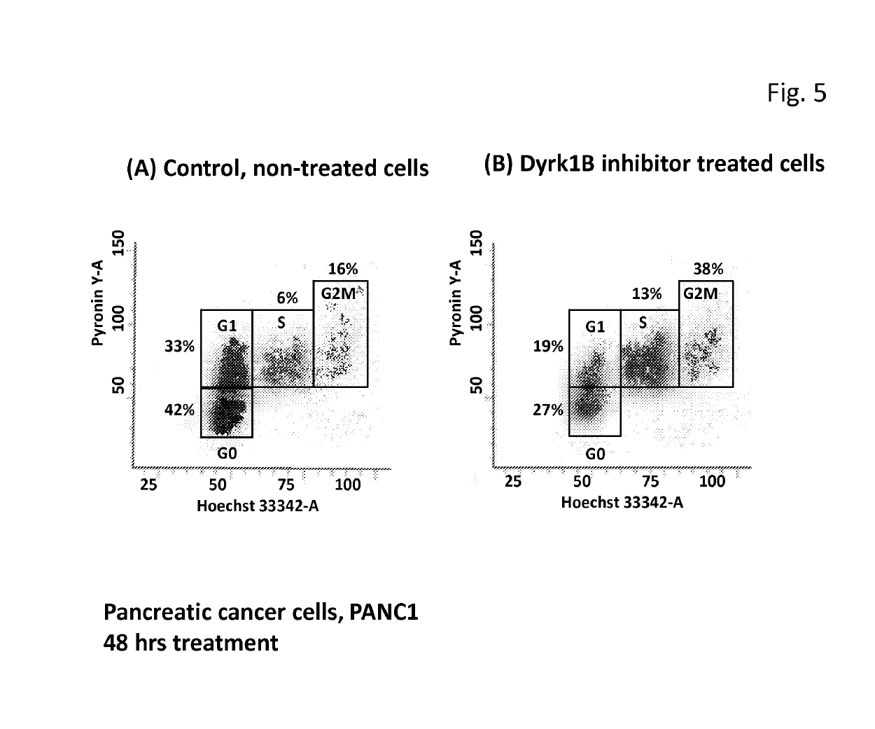

Quiescent cancer cells are insensitive to cytotoxic drugs and radiation therapy, both of which target dividing cells and therefore do not affect cells while in the resting G₀ state. Because chemotherapy and radiation treatments regiments cannot be maintained indefinitely in the body due to their toxic side effects, the surviving quiescent cancer cells can cause cancer recurrence upon re-entry to the cell cycle, the timing of which cannot be predicted. Further, metastatic cancer cells in the bloodstream may experience a period of dormancy or quiescence while they adapt to their new microenvironment (Chaffer, C. L. and Weinberg, R. A. (2011) A perspective on cancer cell metastasis, Science 331, 1559-1564). Quiescent cancer cells degrade their polyribosomes, thus blocking translation and reducing total RNA and protein content. These shrunk metabolically inactive cancer cells may be able to enter the bores of capillaries (approximately 8 μm diameter) whereas cycling cancer cells are usually much larger (20-30 μm). Because cells in G₀ state have total RNA levels lower than those in dividing cells or those preparing for cell cycle division, i.e. cells in G₁, S, or G2/M-phases of the cell cycle, the quiescent cells are readily identified by flow cytometry according to RNA content. In some embodiments of the invention, the presence of quiescent cancer cells is detected by flow cytometry as illustrated in FIG. 5. In other embodiments of the invention, detection of quiescent cancer cells in a cell population comprises determining the fraction of quiescent cancer cells within the cell population.

Cancer cells can either enter an irreversible state before undergoing terminal differentiation, or senescence, or enter a reversible, true quiescent G₀ state or dormant state from which they could resume cycling, like quiescent fibroblasts (Coller, H. A., Sang, L., and Roberts, J. M. (2006) A new description of cellular quiescence, PLoS Biology 4, e83). It has been demonstrated that cultures of serum-starved cancer cells were enriched in G₀ cells (60%-90%) (Deng, X., Ewton, D. Z., and Friedman, E. (2009) DYRK1B maintains the viability of quiescent pancreatic cancer cells by reducing levels of reactive oxygen species, Cancer Research 69, 3317-3324; Jin, K., Ewton, D. Z., Park, S., Hu, J., and Friedman, E. (2009) Mirk regulates the exit of colon cancer cells from quiescence, Journal of Biological Chemistry 284, 22916-22925) and had up to 10-fold higher protein levels of the activated kinase DYRK1B/Mirk and the CDK inhibitor p27kip1, as well as 16-fold higher levels of the retinoblastoma protein family member p130/Rb2 that sequesters the E2F4 transcription factor, thus preventing cells from entering into the cell cycle.

Without being bound by any particular theory, the present invention is based on the discovery that inhibition of the activity of a DYRK1 disrupts cancer cell quiescence, forces cells to re-enter cell cycle, therefore rendering the cancer cells susceptible to radiation therapy or chemotherapy. In example embodiments, the DYRK1 is DYRK1A or DYRK1B.

In an example embodiment, the invention includes methods for treatment of a subject suffering from a neoplasm. These methods include analyzing a sample from the subject, for example a tissue sample, to determine whether a DYRK1 selected from DYRK1A or DYRK1B is expressed in the sample as well as to determine the presence of quiescent neoplastic cells. If quiescent cells are detected and if DYRK1 is expressed or overexpressed in the tissue sample, a modulator of DYRK1 is administered.

Alternately, these methods include analyzing a sample from the subject, for example a tissue sample, to determine the fraction of quiescent neoplastic cells in the cancer cell population as well as to determine the level of expression of a DYRK1 using either or both the determination of the protein expression level or the determination of the number of transcripts (gene copy, cDNA, mRNA, or otherwise suitable). In a certain embodiment, the invention includes collecting a tissue sample from a subject. If a fraction of quiescent cells is determined and if DYRK1 is expressed or overexpressed in the tissue sample, a modulator of DYRK1 is administered.

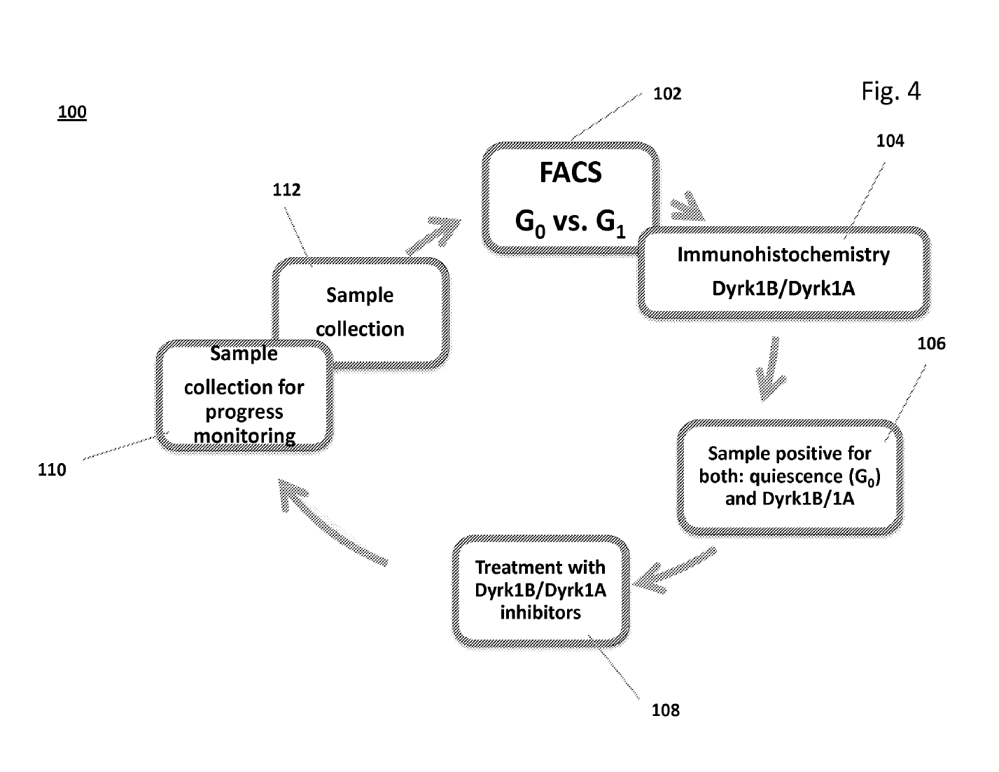

An example embodiment of such a method is shown in FIG. 4. The method 100 includes a step of sample collection 112, followed by the determination of the fraction of the quiescent cells (G₀ to G₁ ratio) in step 102. This determination can be performed by known techniques, for example, by flow cytometry such as fluorescence-activated cell sorting (FACS). The expression level of a DYRK1 is next determined in step 104 by any known technique, e.g., immunohistostaining. Alternatively, the gene copy number or the level of cDNA or mRNA transcripts coding for DYRK1 genes can be quantified by standard PCR or another appropriate technique. If the sample is determined in step 106 to include both the quiescent cells and the cells expressing (or overexpressing a DYRK1), or have elevated gene copy number or the level of cDNA or mRNA transcripts coding for DYRK1 genes, then the subject is treated with an effective amount of a DYRK1 activity modulator, step 108. Method 100 can further include an optional step 110 of collecting another sample at a later time point. Such later sample can be compared to the sample collected in step 112 to monitor the progress of a cancer therapy co-administered with a DYRK1 activity modulator.

The methods of the present invention include treating a subject suffering from a neoplasm, the method comprising administration of a modulator of DYRK1 activity.

In another example embodiment, the invention relates to a modulator of DYRK 1 activity for use in the treatment of a neoplasm in a patient. The treatment includes determining whether a DYRK1 selected from DYRK1A or DYRK1B is expressed in a patient sample and determining whether quiescent neoplastic cells are present in the patient sample. If the patient expresses a DYRK1 selected from DYRK1A or DYRK1B in a patient sample, and if quiescent neoplastic cells are present in the patient sample, an effective amount of the modulator is administered. In a further embodiment, the patient sample is an initial patient sample and a subsequent patient sample is collected after the modulator is administered. The subsequent patient sample is analyzed to determine whether a DYRK1 selected from DYRK1A or DYRK1B is expressed in the subsequent patient sample, as well as to determine a fraction of quiescent neoplastic cells in the subsequent patient sample. If the fraction of quiescent neoplastic cells present in the initial patient sample is greater than the fraction of quiescent neoplastic cells in the subsequent patient sample, and if the patient expresses a DYRK1 selected from DYRK1A or DYRK1B, then a second effective amount of the modulator is administered.

In another example embodiment, the invention further relates to a modulator of DYRK1 activity for use in the treatment neoplasm in a patient, wherein the patient has been determined to express a DYRK1 selected from DYRK1A or DYRK1B in a patient sample and wherein quiescent neoplastic cells have been determined to be present in the patient sample. In certain embodiments, the patient sample is an initial patient sample, a fraction of quiescent neoplastic cells in the initial sample has been determined, and the treatment includes administering a first effective amount of the modulator. In these embodiments, the patient has been determined to express a DYRK1 selected from DYRK1A or DYRK1B in a subsequent patient sample and quiescent neoplastic cells have been determined to be present in the subsequent patient sample. The subsequent patient sample has been collected after administering a first effective amount of the modulator. In further embodiments, the treatment includes administering a second effective amount of the modulator, which is performed after the subsequent patient sample is collected.

In another example embodiment, the invention includes a modulator of DYRK1 activity for use in the treatment of a neoplasm in a patient, wherein the patient has been determined to express a DYRK1 selected from DYRK1A or DYRK1B in an initial patient sample and wherein a fraction of quiescent neoplastic cells has been determined in the initial patient sample, the treatment including administering to the patient a first effective amount of the modulator, and further wherein the patient has been determined to express a DYRK1 selected from DYRK1A or DYRK1B in a subsequent patient sample and wherein a fraction of quiescent neoplastic cells has been determined to be present in the subsequent patient sample, the subsequent patient sample having been collected after administering a first effective amount of the modulator, and wherein the fraction of quiescent neoplastic cells in the initial patient sample is greater than the fraction of quiescent neoplastic cells in the subsequent patient sample.

The modulators of DYRK1 activity described in this invention include those identified by enzymatic activity assay. For example, activity may be measured by using a DYRK1 selected from DYRK1A or DYRK1B, a corresponding substrate for a DYRK1 selected from DYRK1A or DYRK1B, and a modulator of a DYRK1 selected from DYRK1A or DYRK1B. Example assays may be based on phosphorylation of an amino acid residue having a side chain that includes a hydroxyl group, for example, a tyrosine residue, a serine residue, a threonine residue, or a combination of the above residues, on the substrate for a DYRK1 selected from DYRK1A or DYRK1B.

Modulators of DYRK1 activity, for example inhibitors of DYRK1 activity, can be identified by a kinase assay in which the activity of a DYRK1 selected from DYRK1A or DYRK1B is measured in the presence of a potential modulator. Methods for performing this assay include contacting a DYRK1 selected from DYRK1A or DYRK1B with a substrate for a DYRK1 selected from DYRK1A or DYRK1B in a solution, then measuring phosphorylation of the substrate. The solution comprising the DYRK1 selected from DYRK1A or DYRK1B and the substrate for a DYRK1 selected from DYRK1A or DYRK1B is alternately referred to as a reaction mixture. In particular example embodiments, the reaction mixture further comprises a modulator of DYRK1 activity, wherein the modulator may be a DYRK1A specific inhibitor or a DYRK1B specific inhibitor, or alternately may be an inhibitor of both DYRK1A and DYRK1B.

DYRK1B that can be used with the present invention includes any isoform of DYRK1B that is kinase-active, including the 65 kDa isoform (i.e. p65), 69 kDa isoform (i.e. p69), and 75 kDa isoform (i.e. p75).

The reaction mixture used in the methods for assaying the activity of a DYRK1 selected from DYRK1A or DYRK1B further comprises a solvent and a solute. Suitable solutes include adenosine triphosphate, magnesium (II) chloride, a surfactant, a reducing agent, and a buffering agent. In a particular embodiment, the solution contains adenosine triphosphate, magnesium (II) chloride, Brij-35, dithiothreitol, and tris(hydroxymethyl)aminomethane (Tris) hydrochloride. In an example embodiment the solvent is water. The solvent may comprise less than 10% DMSO.

Substrates for DYRK1A and DYRK1B that may be used in the kinase assay include the peptide DYRKtide [RRRFRPASPLRGPPK (SEQ ID NO: 1)], available from AnaSpec, Inc. (34801 Campus Drive, Fremont, Calif. 94555) or dephosphorylated α-casein available from Sigma Aldrich Corporation (3050 Spruce Street, St. Louis, Mo. 63103), or LANCE® Ultra peptide substrates for kinase assays, such as ULight™-Myelin Basic Protein Peptide [VTPRTPPP (SEQ ID NO: 2)], available from Perkin-Elmer, Inc. (940 Winter Street, Waltham, Mass. 02451). In yet further examples, the substrate for a DYRK1 selected from DYRK1A or DYRK1B comprises a fluorescent moiety.

Alternately, the substrate for a DYRK1 selected from DYRK1A or DYRK1B comprises at least one fluorescent moiety having a fluorescent emission between about 200 nanometers and 900 nanometers. Examples of fluorescent moieties that may be used with the substrate include any of the fluorescein family of dyes, for example 5-FAM, or any of the rhodamine family of dyes such as 5-TAMRA, or any other dyes with appropriate excitation and emission wavelengths. If the substrate comprises a fluorescent moiety having a fluorescent emission between about 200 nanometers and 900 nanometers, then the measurement of the substrate may be conveniently performed by measuring the optical fluorescence of the peptide.

When the substrate is phosphorylated, the mass, and charge, and mass to charge ratio of the substrate are altered. The substrate may be measured by mass spectrometry or electrophoresis. The quantities of phosphorylated and unphosphorylated substrate may be measured using mass spectrometry, electrophoresis, X-ray fluorescence, or optical fluorescence.

The source of phosphate, for example adenosine triphosphate, may comprise radioactive phosphorus. In an example embodiment of a method for assaying the activity of a DYRK1, a peptide substrate is reacted with the kinase enzyme in the presence of a form of adenosine triphosphate, which is capable of donating a radioactive phosphate moiety to the peptide during the kinase reaction, and the extent of the progression of the reaction is measured using a radiometric method such as phosphoimager, scintillation counting, and the like.

In further methods for identifying modulators of DYRK1 by enzymatic assay, a peptide substrate is modified to contain a fluorescent moiety and is reacted with a kinase enzyme, and the extent of phosphorylation is monitored using fluorescence anisotropy, fluorescence resonance energy transfer, time-resolved fluorescence energy transfer, and the like.

Alternately, a peptide substrate is modified with a fluorescent moiety that is reacted with a kinase enzyme, with the extent of phosphorylation monitored using differential chromatographic mobility, or alternately differential electrophoretic mobility.

Alternately, a peptide substrate is reacted with a kinase enzyme, with the extent of phosphorylation monitored using differential electrophoretic mobility or X-ray fluorescence.

Certain example methods for identifying modulators of DYRK1 utilize analysis by Fluorescence Resonance Energy Transfer (FRET) or time-resolved fluorescence resonance transfer (TR-FRET), or variations thereof. For example, LanthaScreen™ Kinase Activity Assays (Invitrogen, Life Technologies 3175 Staley Road, Grand Island, N.Y. 14072) may be used to detect inhibitors of a DYRK1 selected from DYRK1A or DYRK1B. The components of the reaction mixture in this assay include a DYRK1 selected from DYRK1A or DYRK1B, ATP, buffer components and additives, a substrate molecule such as a LANCE® Ultra peptide substrate capable of being phosphorylated by DYRK1, wherein the phosphorylated substrate molecule is labeled with a fluorescence acceptor such as AlexaFluor® 647, and an antibody capable of binding selectively to the phosphorylated substrate, wherein the antibody is labeled with terbium or another group capable of fluorescence energy transfer to the AlexaFluor® 647 or another appropriate acceptor. FRET may be detected by fluorescence microscopy and additional methods known to those of ordinary skill in the art. The decrease in the FRET signal is utilized to detect and measure inhibition of the kinase activity by an inhibitor.

Certain example methods for identifying modulators of DYRK1 utilize indirect measures of kinase activity. For example, ADP-Glo™ Kinase Assay available from Promega Corporation (2800 Woods Hollow Road Madison, Wis. 53711 USA) utilizes luminescent quantification of adenosine diphosphate (ADP) produced during a kinase reaction. The components of the reaction mixture in this assay include a DYRK1 selected from DYRK1A or DYRK1B, ATP, buffer components and additives, and may include a substrate molecule such as DYRKtide capable of being phosphorylated by DYRK1. The assay is performed in two steps; first, after the kinase reaction, an equal volume of ADP-Glo™ Reagent is added to terminate the kinase reaction and deplete the remaining ATP. Second, the Kinase Detection Reagent is added to simultaneously convert ADP to ATP and allow the newly synthesized ATP to be measured using a luciferase/luciferin reaction. The light generated is measured by a luminometer or another appropriate instrument. The decrease in luminescence is correlated to the inhibition of the kinase activity by an inhibitor.

Certain example methods for identifying and characterizing modulators of DYRK1 rely on the detection of binding of modulators to DYRK1 and utilize analysis by Fluorescence Resonance Energy Transfer (FRET) or time-resolved fluorescence energy transfer (TR-FRET) to detect and characterize inhibitors of a DYRK1 selected from DYRK1A or DYRK1B. For example, a LanthaScreen™ Eu kinase binding assay (Invitrogen, Life Technologies 3175 Staley Road, Grand Island, N.Y. 14072) may be used to detect and characterize inhibitors of a DYRK1 selected from DYRK1A or DYRK1B. The components of the reaction mixture in this assay include a DYRK1 selected from DYRK1A or DYRK1B, ATP, buffer components, a tracer molecule capable of binding to the active site of the DYRK1 wherein the tracer molecule is labeled with a fluorescence acceptor such as AlexaFluor® 647, and an antibody capable of binding to the kinase, wherein the antibody is labeled with a fluorescence donor group such as europium. Simultaneous binding of the Eu-labeled antibody and the AlexaFluor®-labeled tracer to a DYRK1 causes FRET from the fluorescence donor, for example Eu, to the fluorescence acceptor, for example AlexaFluor®. FRET may be detected by fluorescence microscopy and additional methods known to those of ordinary skill in the art. The disruption of a FRET signal is utilized to detect inhibition of the binding of the tracer by an inhibitor.

Example inhibitors of DYRK1A activity include small molecule heterocyclic compounds TBB (CAS: [17374-26-4]):

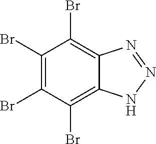 [SMILES: Brc1c(Br)c(Br)c2[nH]nnc2c1Br]

### and (S)-CR8 (CAS: [1084893-56-0]):

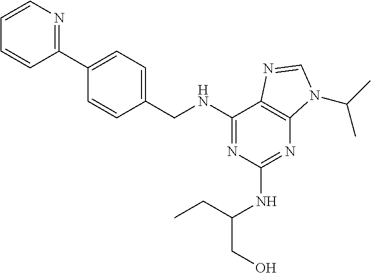 [SMILES: CCC(CO)Nc1nc(NCc2ccc(-c3ccccn3)cc2)c2ncn(C(C)C)c2n1]

both available from Santa Cruz Biotechnology, Inc. (10410 Finnell Street Dallas, Tex. 75220).

Example modulators of DYRK1B activity include peptidic DYRK1B inhibitors 4IPhe-Cys-Thr-Asn-Cys-Glu-Thr-Gly-Cys-BrPhe (SEQ ID NO: 3), BrTyr-Cys-Ala-Ser-Asp-Cys (SEQ ID NO: 4), BrPhe-Ile-Asn-Thr-Lys-Met-Met-Trp-4IPhe (SEQ ID NO: 5), BrPhe-His-Ala-Thr-Lys-Phe-Ala-Ala-4IPhe (SEQ ID NO: 6), and BrPhe-Ile-Trp-Tyr-Arg-Gln-Asn-Val-4IPhe (SEQ ID NO: 7), wherein BrPhe is a bromophenylalanine, for example 4-bromophenylalanine, 3-bromophenylalanine, or 2-bromophenylalanine; 4IPhe refers to 4-iodophenylalanine; BrTyr refers to 3,5-dibromotyrosine; Ala refers to Alanine; Arg refers to Arginine; Asn refers to Asparagine; Asp refers to Aspartic acid; Cys refers to Cysteine; Glu refers to Glutamic acid; Gln refers to Glutamine; Gly refers to Glycine; His refers to Histidine; Ile refers to Isoleucine; Leu refers to Leucine; Lys refers to Lysine; Met refers to Methionine; Phe refers to Phenylalanine; Pro refers to Proline; Ser refers to Serine; Thr refers to Threonine; Trp refers to Tryptophan; Tyr refers to Tyrosine; and Val refers to Valine.

In alternate embodiments, the modulator of DYRK1B activity is a small molecule DYRK1B inhibitor, for example the compound of the Formula:

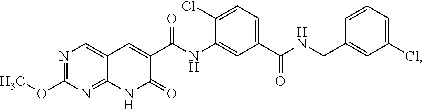 [SMILES: COc1ncc2cc(C(=O)Nc3cc(C(=O)NCc4cccc(Cl)c4)ccc3Cl)c(=O)[nH]c2n1]

and pharmaceutically acceptable salts thereof.

In yet further embodiments of the invention, the inhibitor of a DYRK1 selected from DYRK1A or DYRK1B is an antibody that has been raised against a DYRK1 enzyme. The antibody can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein also encompasses functional fragments of antibodies, including fragments of chimeric, humanized, primatized, veneered, or single chain antibodies. Functional fragments include antigen-binding fragments which bind to a DYRK1. Such fragments can be produced by enzymatic cleavage or by recombinant techniques. For example, papain, pepsin, or other protease with the requisite substrate specificity can be used to generate fragments. Antibodies can also be produced in a variety of truncated forms using antibody genes in which one or more stop codons has been introduced upstream of the natural stop site.

Further examples of DYRK1B modulators useful for practicing methods of the present invention include those disclosed by U.S. Patent Application Publication Nos. 2012/0184542 and 2012/0184548.

In some embodiments of the invention, the method for treating a subject suffering from a neoplasm further comprises administering to the subject an effective amount of at least one additional therapeutic agent. In certain embodiments, the invention relates to a modulator of DYRK1 activity for use in the treatment of a neoplasm, the treatment further comprises administering to the patient an effective amount of at least one additional therapeutic agent.

In certain embodiments, the at least one additional therapeutic agent is a chemotherapeutic agent. A chemotherapeutic agent may be selected from anti-metabolites, alkylating agents, anthracyclines, plant alkaloids, topoisomerase inhibitors, cytostatic agents, cytotoxic agents, immunological modifiers such as interferons, interleukins, growth hormones or other cytokines, or other chemotherapeutic agents. Examples of chemorethapeutic agents suitable for using in the methods described herein are provided above. The chemotherapeutic agent can be administered before, simultaneously with, or after the modulator of DYRK1 activity.

In alternative embodiments of the invention, the method further comprises administering to the subject an effective amount of radiation therapy in conjunction with the modulator of DYRK1. Alternatively, in embodiments of the invention relating to a modulator of DYRK1 activity for use in the treatment of a neoplasm, the treatment further comprises administering to the patient an effective amount of radiation therapy in conjunction with the modulator of DYRK1 activity. Radiation therapy can be administered before, simultaneously with, or after the modulator of DYRK1 activity.

In alternative embodiments of the invention, the modulator of DYRK1 activity is administered in conjunction with an immunotherapeutic agent, an antiangiogenic agent, or before or after surgical resection of a cancer. Any of these agents can be administered before, simultaneously with, or after the modulator of DYRK1 activity.

In some embodiments of the invention, the modulator of DYRK1 activity is administered to a subject who has undergone surgical resection of a cancer. In example embodiments of the invention, the modulator of DYRK1 activity is a specific DYRK1B inhibitor. In alternative embodiments, the modulator is a specific DYRK1A inhibitor.

In example embodiments of the invention, wherein the invention is a method for treating a subject suffering from a neoplasm, or alternately wherein the invention is a modulator for use in the treatment of a neoplasm in a patient, the neoplasm is a solid cancer that may include a pancreatic cancer, a breast cancer, a colon cancer, a rectal cancer, a lung cancer, an ovarian cancer, a testicular cancer, or an osteosarcoma. In another embodiment of the invention, the neoplasm is a xenograft. In alternative embodiments of the invention, the neoplasm is a hematopoietic cancer. In further embodiments of the invention, the hematopoietic cancer is selected from leukemia or lymphoma. In some embodiments of the invention, the leukemia is acute myelogenous leukemia or chronic myelogenous leukemia.

In example embodiments of the invention, collecting a sample from the subject includes collecting a blood sample or collecting a tissue biopsy. The subject in the invention may be human or a non-human animal.

In some embodiments of the invention determining the presence of quiescent cancer cells includes defining the distribution of the cellular population within particular stages of the cell cycle and determining whether quiescent cancer cells are or are not present. In an alternative embodiment of the invention, determining the presence of quiescent cancer cells includes determining the fraction of quiescent cancer cells within a cell population. In some embodiments of the invention, the presence of quiescent cancer cells in a cellular population is determined by any technique known in the art, such as, for example, fluorescence activated cell sorting (FACS), which is a specialized type of Flow Cytometry analysis by content of DNA and RNA. In an example embodiment, the fraction of quiescent cancer cells is determined by the distribution content of DNA and RNA in a specific stage of cell cycle as determined by FACS analysis. As illustrated in FIG. 5, serum-starved pancreatic cancer cells (PANC-1) had a large fraction (42%) of cells in the G₀ state. After treatment with a DYRK1B inhibitor, the fraction of cells in the G₀ state reduced to 27%.

In some embodiments of the invention, the determination of the expression level of a DYRK1 selected from DYRK1A or DYRK1B is made by immunohistostaining. In others, the determination of the gene copy number or the level of cDNA or mRNA transcripts coding for DYRK1 genes is made by PCR or another appropriate technique.

The methods presented herein are useful in the treatment of a subject suffering from a neoplasm in any stage of the disease. The methods described herein can be used for identifying and selecting the most suitable treatment for the tested individual, reducing the maximum dose of the chemotherapeutic agent needed, and preventing cancer growth, resistance, metastasis, and recurrence. Upon identification of a cancer type with respect to the ability of its cells to resist treatment through residence in a quiescent state, a treatment can be adjusted to mitigate such resistance.

The methods disclosed herein can be used to determine the most suitable therapy for the treatment of various types of neoplasms. The neoplasm can be a solid cancer including pancreatic cancer, breast cancer, colon cancer, rectal cancer, lung cancer, ovarian cancer, or osteosarcoma. The neoplasm can be a hematopoietic cancer, such as leukemia or lymphoma. Examples of a hematopoietic cancers include acute myelogenous leukemia or chronic myelogenous leukemia.

The methods disclosed herein can be used for adjusting a therapy for treating a subject suffering from a neoplasm. In one example embodiment, the present invention is a method for treating a subject suffering from a neoplasm, comprising: (a) determining the expression level of a DYRK1 selected from DYRK1A or DYRK1B within a cell population in a first sample of quiescent neoplastic cells and/or determining the overall fraction of quiescent neoplastic cells in a first sample; (b) determining expression level of a DYRK1 selected from DYRK1A or DYRK1B within a cell population in a second sample of quiescent neoplastic cells, and/or determining the overall fraction of quiescent neoplastic cells in a second sample, wherein the first sample is collected at a first time point, and the second sample is collected at a second time point that is later than the first time point; and (c) if the expression of the DYRK1 is confirmed through either or all protein, mRNA, cDNA, and/or gene copy number measurements, and moreover if the expression increases or remains static from the first time point to the second time point, then administering an effective amount of a modulator of DYRK1 activity in conjunction with an effective amount of an additional therapeutic agent.

The methods disclosed herein may be used to monitor the effectiveness of treatment of cancers that express DYRK1B. In example embodiments of the invention, the methods provide for augmenting an existing cancer treatment regimen based on the identification of a patient's cancer cell population as expressing or overexpressing DYRK1B.

In another example embodiment, the present invention further relates to a method for reducing the fraction of quiescent cancer cells in a cell population, comprising incubating the cell population with a modulator of DYRK1 activity for a period of time sufficient to reduce the fraction of quiescent cancer cells in the cell population. In some embodiments of the invention, the cell population is incubated with the modulator from about 1 hour to approximately 96 hours. In some embodiments, the incubation time is from approximately 18 hours to approximately 120 hours or more. The modulator of DYRK1 activity is an inhibitor or an activator of DYRK1 activity. In some embodiments of the invention, the modulator of DYRK1 activity is a specific DYRK1A inhibitor or a specific DYRK1B inhibitor. In another embodiment of the invention, the modulator is a specific DYRK1B inhibitor.

In certain embodiments, the present invention is a method of treating a subject suffering from a disorder selected from Down Syndrome or early onset Alzheimer's disease. Such a method comprises determining the expression level of a DYRK1 selected from DYRK1A or DYRK1B within a cell population in a sample collected from the subject, and if DYRK1 is expressed in the sample, administering to the subject an effective amount of a modulator of DYRK1 activity. Simultaneously, the level of quiescent cell population and the effect of DYRK1 modulator on the ability of cells to remain in quiescent state might be monitored.

The practice of this invention will employ, unless otherwise indicated, conventional methods of chemistry, biochemistry, molecular biology and pharmacology, within the skill of the art. Such techniques are explained fully in the literature. See, e.g. Remington's Pharmaceutical Sciences 18th Edition (DR, 1985). Short Protocols in Molecular Biology (Frederick M. Ausubel (Editor), 2002), Molecular Biology Techniques An Intensive Laboratory Course (Walt Ream, 1998). Although methods and materials similar or equivalent to those described herein can be used in the practice or testing of the present disclosure, suitable methods and materials are described below. In case of conflict, the present specification, including explanation of terms, will control. In addition, the materials, methods, and examples are illustrative only and not intended to be limiting. Furthermore, unless otherwise required by context, singular terms shall include pluralities and plural terms shall include the singular.

### EXEMPLIFICATION

The following examples are not intended to be limiting. Those of skill in the art will, in light of the present disclosure, appreciate that many changes can be made in the specific materials and which are disclosed and still obtain a like or similar result without departing from the spirit and scope of the invention.

### Example 1

### DYRK1B Kinase Inhibitor Blocks Accumulation of Quiescent Cancer Cells

It was suggested that the role of DYRK1B kinase is to maintain cancer cells in quiescent state. The ability of DYRK1B kinase to allow cycling cancer cells to enter a reversible quiescent state has been analyzed in pancreatic cancer cells, Panc-1. Panc-1 cells demonstrated elevated expression levels of DYRK1B kinase (Deng, X., Ewton, D. Z., Li, S., Naqvi, A., Mercer, S. E., Landas, S., and Friedman, E. (2006) The kinase Mirk/DYRK1B mediates cell survival in pancreatic ductal adenocarcinoma, Cancer Research 66, 4149-4158); (Karhu, R., Mahlamaki, E., and Kallioniemi, A. (2006) Pancreatic adenocarcinoma—genetic portrait from chromosomes to microarrays, Genes, Chromosomes and Cancer 45, 721-730).

Panc-1 cells were forced into the quiescent state by serum deprivation for 48 hours. Panc-1 were cultured in DMEM+0.2% FBS for 48 hours. Next the cells were switched to fresh media DMEM+10% FBS (panel A) or to fresh media DMEM+1-% FBS supplemented with 0.25 μM DYRK1B inhibitor R (panel B). As a result of this serum deprivation, 42% of cells were identified in G₀ quiescent state, as depicted in FIG. 5A. The percentage of quiescent cells was identified by flow cytometry FACS analysis.

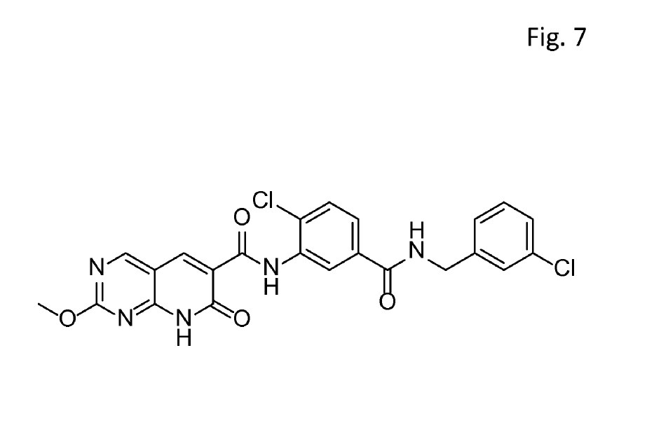

Next, a DYRK1B small molecule inhibitor R, pictured in FIG. 7, was applied to inhibit the activity of DYRK1B kinase, and the percentage of quiescent cells was again analyzed. Parallel Panc-1 cultures were treated with the DYRK1B kinase inhibitor R. FACS analysis revealed that only 27% of Panc-1 cells were in G₀ (FIG. 5B). Upon inhibition of the activity of DYRK1B kinase, pancreatic cancer cells left the G₀ quiescent state and progressed into cell cycle. While the number of quiescent cells had been reduced to 27% in the presence of DYRK1B inhibitor R, the number of cells in S-phase of cell cycle doubled (13%) as compared to control cells (6% in FIG. 5A). DYRK1B kinase activity was necessary for Panc-1 pancreatic cancer cells to accumulate in a quiescent state. The distribution of cellular population within the cell cycle (i.e. G₀, G₁, S and G₂/M populations) was analyzed by two-parameter flow cytometry. Specifically, the incorporation of Pyronin Y and Hoechst staining correlated to RNA and DNA content distribution, respectively. (Ewton, D. Z., Hu, J., Vilenchik, M., Deng, X., Luk, K. C., Polonskaia, A., Hoffman, A. F., Zipf, K., Boylan, J. F., and Friedman, E. A. (2011) Inactivation of mirk/dyrk1b kinase targets quiescent pancreatic cancer cells, Molecular Cancer Therapeutics 10, 2104-2114).

The presence of DYRK1B inhibitor R blocked additional Panc-1 cells from entering quiescence.

### Example 2

### Inhibition of DYRK1B Kinase Activity Results in Blocking Cancer Cells Ability to Enter G₀ State and Allowing Cells Re-Entry into the Cell Cycle

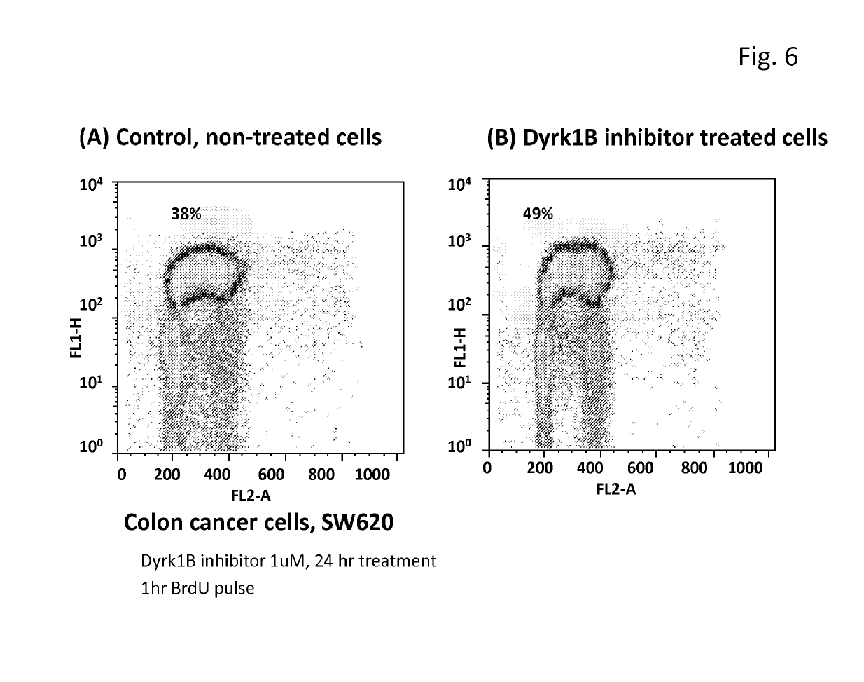

An experiment was run to determine if inhibition of DYRK1B activity would facilitate cells escaping quiescence and result in surge of proliferating cells, utilizing small molecule inhibitor R. Cycling, log-phase cells from colon cancer cell line SW620 were treated with 1 μM of DYRK1B inhibitor R for 24 hours with 1 hour pulse of BrdU before analysis by flow cytometry after propidium iodine staining of DNA. 38% of the cellular population was detected in the S-phase of the cell cycle. Parallel cultures were treated with the small molecule DYRK1B inhibitor R for 24 hours followed by identical FACS analysis (1-hour BrdU pulse followed by specific mAB-FITC and PI staining). Flow cytometry analysis showed that the fraction of S-phase cells was increased 29% as the result of inhibition of DYRK1B activity in SW620 cells (FIG. 6B) as compared to the non-treated, control SW620 cells (FIG. 6A). Increase in S-phase population of cells following DYRK1B inhibition up to 49% (FIG. 6B) as compared to 38% (FIG. 6A), indicated that these cells had an increased amount of DNA synthesis, and thus had re-entered the active cell cycle as a consequence of the inhibition of the DYRK1B activity.

While this invention has been particularly shown and described with references to example embodiments thereof, it will be understood by those skilled in the art that various changes in form and details may be made therein without departing from the scope of the invention encompassed by the appended claims.

SEQUENCE LISTING

| | |
|---|---|
| <110> | FELICITEX THERAPEUTICS, INC. |
| <120> | TREATMENT OF CANCER BY TARGETING QUIESCENT |
| | CANCER CELLS |
| <130> | FT1-US1 |
| <150> | PCT/US13/64345 |
| <151> | 2010 Oct. 10 |
| <160> | 7 |
| <170> | PatentIn version 3.5 |
| <210> | 1 |
| <211> | 15 |
| <212> | PRT |
| <213> | Artificial Sequence |
| <220> | |
| <223> | Artificial sequence is synthesized |
| <400> | 1 |

Arg Arg Arg Phe Arg Pro Ala Ser Pro Leu Arg Gly Pro Pro Lys
1 5 10 15

| | |
|---|---|
| <210> | 2 |
| <211> | 8 |
| <212> | PRT |
| <213> | Artificial Sequence |
| <220> | |
| <223> | Artificial sequence is synthesized |
| <400> | 2 |

Val Thr Pro Arg Thr Pro Pro Pro
1 5

| | |
|---|---|
| <210> | 3 |
| <211> | 10 |
| <212> | PRT |
| <213> | Artificial Sequence |
| <220> | |
| <223> | Artificial sequence is synthesized |
| <220> | |
| <221> | MISC_FEATURE |
| <222> | (1)..(1) |
| <223> | 4-iodophenylalanine |
| <220> | |
| <221> | MISC_FEATURE |
| <222> | (10)..(10) |
| <223> | bromphenylalanine, for example 4-bromophenylalanine, |
| | 3-bromophenylalanine, or 2 bromophenylalanine |
| <400> | 3 |

Xaa Cys Thr Asn Cys Glu Thr Gly Cys Xaa
1 5 10

| | |
|---|---|
| <210> | 4 |
| <211> | 6 |
| <212> | PRT |
| <213> | Artificial Sequence |
| <220> | |
| <223> | artificial sequence is synthesized |
| <220> | |
| <221> | MISC_FEATURE |
| <222> | (1)..(1) |
| <223> | 3,5-dibromotyrosine |
| <400> | 4 |

Xaa Cys Ala Ser Asp Cys
1 5

| | |
|---|---|
| <210> | 5 |
| <211> | 9 |
| <212> | PRT |
| <213> | Artificial Sequence |
| <220> | |
| <223> | artificial sequence is synthesized |
| <220> | |
| <221> | MISC_FEATURE |
| <222> | (1)..(1) |
| <223> | Bromophenylalanine, for example 4-bromophenylalanine, |
| | 3-bromophyenylalanine, or 2-bromophenylalanine |
| <220> | |
| <221> | MISC_FEATURE |
| <222> | (9)..(9) |
| <223> | 4-iodophenylalanine |
| <400> | 5 |

Xaa Ile Asn Thr Lys Met Met Trp Xaa
1 5

| | |
|---|---|
| <210> | 6 |
| <211> | 9 |
| <212> | PRT |
| <213> | Artificial Sequence |
| <220> | |
| <223> | Artificial sequence is synthesized |
| <220> | |
| <221> | MISC_FEATURE |
| <222> | (1)..(1) |
| <223> | bromophenylalanine, for example 4-bromophenylalanine, |
| | 3-bromophenylalanine, or 2-bromophenylalanine |
| <220> | |
| <221> | MISC_FEATURE |
| <222> | (9)..(9) |
| <223> | 4-iodophenylalanine |
| <400> | 6 |

Xaa His Ala Thr Lys Phe Ala Ala Xaa
1 5

| | |
|---|---|
| <210> | 7 |
| <211> | 9 |
| <212> | PRT |
| <213> | Artificial Sequence |
| <220> | |
| <223> | Artificial sequence is synthesized |
| <220> | |
| <221> | MISC_FEATURE |
| <222> | (1)..(1) |
| <223> | bromophenylalanine, for example 4-bromophenylalanine, |
| | 3-bromphenylalanine, or 2-bromophenylalanine |
| <220> | |
| <221> | MISC_FEATURE |
| <222> | (9)..(9) |
| <223> | 4-iodophenylalanine |
| <400> | 7 |

Xaa Ile Thr Tyr Arg Gln Asn Val Xaa
1 5

What is claimed is:

1-47. (canceled)

48. A method for treating a patient suffering from a neoplasm, the method comprising:
(a) determining the level of expression of DYRK1 selected from DYRK1A or DYRK1B in a blood or tissue biopsy sample collected from a patient;
(b) determining whether quiescent neoplastic cells are present in the patient sample; and
(c) if the DYRK1 is expressed in the sample and if quiescent neoplastic cells are present in the sample, then administering to the patient an effective amount of a modulator of DYRK1 activity.

49. The method of claim 48, wherein determining whether quiescent neoplastic cells are present comprises utilizing flow cytometry to determine the distribution of the cellular population from the sample.

50. The method of claim 48, wherein determining the level of expression of DYRK1 is done by immunohistostaining or utilizing PCR.

51. The method of claim 48, further comprising administering to the patient an effective amount of at least one additional therapeutic agent.

52. The method of claim 48, further comprising administering to the patient an effective amount of radiation therapy.

53. The method of claim 48, wherein the neoplasm is a solid cancer selected from a pancreatic cancer, a breast cancer, a colon cancer, a rectal cancer, a lung cancer, an ovarian cancer, or an osteosarcoma.

54. The method of claim 48, wherein the modulator of DYRK1 activity is administered to a subject who underwent surgical resection of a solid cancer.

55. The method of claim 48, wherein the neoplasm is a hematopoietic cancer.

56. The method of claim 48, wherein the modulator of DYRK1 activity is a specific DYRK1A inhibitor.

57. The method of claim 48, wherein the modulator of DYRK1 activity is a specific DYRK1B inhibitor.

58. The method of claim 48, wherein the modulator has the structure of the formula: 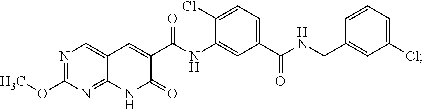 [SMILES: COc1ncc2cc(C(=O)Nc3cc(C(=O)NCc4cccc(Cl)c4)ccc3Cl)c(=O)[nH]c2n1]
or a pharmaceutically acceptable salt thereof.

59. The method of claim 48, wherein the modulator has the structure of the formula: 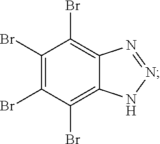 [SMILES: Brc1c(Br)c(Br)c2[nH]nnc2c1Br]
or a pharmaceutically acceptable salt thereof.

60. The method of claim 48, wherein the modulator has the structure of the formula: 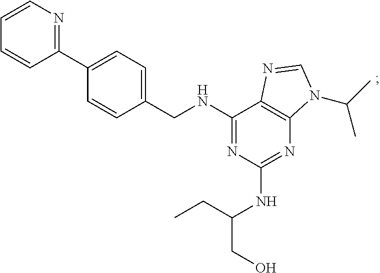 [SMILES: CCC(CO)Nc1nc(NCc2ccc(-c3ccccn3)cc2)c2ncn(C(C)C)c2n1]
or a pharmaceutically acceptable salt thereof.

61. The method of claim 48, wherein the modulator is a peptide consisting of the amino acid sequence selected from the group consisting of SEQ ID NOS: 3-7 and pharmaceutically acceptable salt thereof.

62. A method for treating a patient suffering from a neoplasm, the method comprising:
(a) determining whether a DYRK1 selected from DYRK1A or DYRK1B is expressed in an initial patient sample, the initial patient sample collected from a patient;
(b) determining a fraction of quiescent neoplastic cells present in the initial patient sample;
(c) if the DYRK1 is expressed in the initial patient sample and if the fraction of quiescent neoplastic cells in the initial patient sample is greater than zero, then administering to the patient a first effective amount of a modulator of DYRK1 activity;
(d) determining whether the DYRK1 selected from DYRK1A or DYRK1B is expressed in a subsequent patient sample, the subsequent patient sample collected from the patient after administering to the patient the first effective amount of the modulator of DYRK1 activity;
(e) determining a fraction of quiescent neoplastic cells in the subsequent patient sample; and
(f) if DYRK1 is expressed in the subsequent patient sample and if the fraction of quiescent neoplastic cells in the initial patient sample is greater than the fraction of quiescent neoplastic cells in the subsequent patient sample, then administering to the patient a second effective amount of a modulator of the DYRK1 activity.

63. The method of claim 62, further comprising administering to the subject an effective amount of at least one additional therapeutic agent.

64. A modulator of DYRK1 activity for use in the treatment of a neoplasm in a patient, the treatment comprising:
(a) determining whether a DYRK1 selected from DYRK1A or DYRK1B is expressed in a patient sample;
(b) determining whether quiescent neoplastic cells are present in the patient sample;
(c) selecting for treatment a patient expressing a DYRK1 selected from DYRK1A or DYRK1B in a patient sample, wherein quiescent neoplastic cells are present in the patient sample; and
(d) administering to the patient an effective amount of the modulator.

65. The modulator of claim 64, wherein:
the patient sample is an initial patient sample;
the effective amount of the modulator is a first effective amount of a modulator;
wherein step (b) further comprises determining a fraction of quiescent neoplastic cells in the initial patient sample; and
wherein the treatment further comprises:
(e) determining whether a DYRK1 selected from DYRK1A or DYRK1B is expressed in a subsequent patient sample, wherein the subsequent patient sample is collected from the patient after step (d) is performed;
(f) determining a fraction of quiescent neoplastic cells in the subsequent patient sample;
(g) selecting for treatment a patient expressing a DYRK1 selected from DYRK1A or DYRK1B in the subsequent patient sample, wherein a fraction of quiescent neoplastic cells present in the initial patient sample is greater than the fraction of quiescent neoplastic cells in the subsequent patient sample; and
(h) administering to the patient a second effective amount of the modulator.

66. The modulator of claim 64, wherein the modulator is a peptide consisting of the amino acid sequence selected from the group consisting of SEQ ID NOS: 3-7 and pharmaceutically acceptable salt thereof.

67. The modulator of claim 64, wherein the modulator has the structure of the formula: 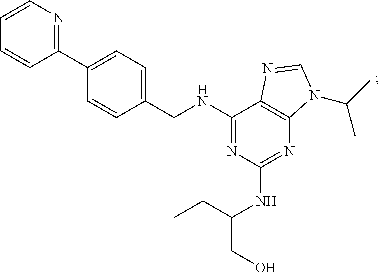 [SMILES: CCC(CO)Nc1nc(NCc2ccc(-c3ccccn3)cc2)c2ncn(C(C)C)c2n1]
or a pharmaceutically acceptable salt thereof.


SEQUENCE LISTING (7 sequences)

SEQ ID NO: 1 - protein, 15 aa, Artificial Sequence
  Artificial sequence is synthesized
RRRFRPASPLRGPPK

SEQ ID NO: 2 - protein, 8 aa, Artificial Sequence
  Artificial sequence is synthesized
VTPRTPPP

SEQ ID NO: 3 - protein, 10 aa, Artificial Sequence
  Artificial sequence is synthesized
XCTNCETGCX

SEQ ID NO: 4 - protein, 6 aa, Artificial Sequence
  artificial sequence is synthesized
XCASDC

SEQ ID NO: 5 - protein, 9 aa, Artificial Sequence
  artificial sequence is synthesized
XINTKMMWX

SEQ ID NO: 6 - protein, 9 aa, Artificial Sequence
  Artificial sequence is synthesized
XHATKFAAX

SEQ ID NO: 7 - protein, 9 aa, Artificial Sequence
  Artificial sequence is synthesized
XITYRQNVX